(19) United States
(12) Patent Application Publication
(10) Pub. No.: US 2020/0188137 A1
(43) Pub. Date: Jun. 18, 2020

(54) ARTIFICIAL MUSCLE ACTUATOR TETHERING LOOPS

(71) Applicant: Lintec of America, Inc., Richardson, TX (US)

(72) Inventors: Marcio Dias LIMA, Richardson, TX (US); Marilu GUERRERO, Richardson, TX (US)

(73) Assignee: Lintec of America, Inc., Richardson, TX (US)

(21) Appl. No.: 16/607,844

(22) PCT Filed: Jul. 3, 2018
(86) PCT No.: PCT/US2018/040727

§ 371 Date: Oct. 24, 2019

Related U.S. Application Data
(60) Provisional application No. 62/528,328, filed on Jul. 3, 2017.

(51) Int. Cl. A61F 2/50; A61F 2/08; D02G 3/44; D02G 3/02

(52) U.S. Cl. CPC A61F 2/5044; A61F 2/0811; D02G 3/448; D02G 3/02; A61F 2002/5066; A61F 2240/001; D10B 2321/08; D10B 2101/122; D10B 2331/02; D10B 2331/10; D10B 2321/042; D10B 2321/021; A61F 2220/0041

(57) ABSTRACT

A method of manufacturing an artificial muscle fiber device includes: tethering an artificial muscle fiber around one or more shape-setting pieces; annealing the artificial muscle fiber so that the artificial muscle fiber will retain specific shapes established by the shape-setting pieces; and removing the shape-setting pieces from the artificial muscle fiber.

### CROSS REFERENCE TO RELATED APPLICATIONS

This Application claims priority to U.S. Provisional Application No. 62/528,328 filed on Jul. 3, 2017, the contents of which are incorporated by reference in their entirety.

### BACKGROUND OF INVENTION

Thermally driven torsional actuators based on twisted polymeric and carbon nanotube (CNT) fibers and yarns have a wide range of applications. Artificial muscle actuators comprising twisted and/or coiled polymers have the advantage of low cost, high production volume, and design simplicity. Artificial muscle actuators may have advantages over small motors because of the greatly simplified engineering and lower product costs.

### SUMMARY OF INVENTION

In one aspect, embodiments disclosed herein relate to a method of manufacturing an artificial muscle fiber device. The method includes: tethering an artificial muscle fiber around one or more shape-setting pieces; annealing the artificial muscle fiber so that the artificial muscle fiber will retain specific shapes established by the shape-setting pieces; and removing the shape-setting pieces from the artificial muscle fiber.

In another aspect, embodiments disclosed herein relate to an artificial muscle actuator device that includes a first artificial muscle fiber segment. The first artificial muscle fiber segment includes a first specific shape established by first segment shape-setting pieces, and by annealing the first specific shape. The artificial muscle actuator device further includes a first fastener and a first holder. The first fastener fastens the first specific shape to the first holder.

Other aspects and advantages of one or more embodiments disclosed herein will be apparent from the following description and the appended claims.

### BRIEF DESCRIPTION OF DRAWINGS

FIGS. 1A-1D show implementation examples in accordance with one or more embodiments of the invention.

FIG. 2 shows a flowchart in accordance with one or more embodiments of the invention.

FIG. 3 shows an implementation example in accordance with one or more embodiments of the invention.

FIG. 4 shows an implementation example in accordance with one or more embodiments of the invention.

FIG. 5 shows an implementation example in accordance with one or more embodiments of the invention.

### DETAILED DESCRIPTION

Specific embodiments of the invention will now be described in detail with reference to the accompanying figures. Like elements in the various figures are denoted by like reference numerals for consistency.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

In general, embodiments of the invention provide an actuator device and a method of manufacturing the actuator device using artificial muscle fibers. An artificial muscle fiber may be a fiber that is made of twist-spun polymeric fibers or nanofiber yarns. The fibers in the artificial muscle fiber may have been twisted to create a specific net bias angle with respect to the length of the artificial muscle fiber.

Upon powering an artificial muscle fiber (i.e., delivering energy to the artificial muscle fiber), the artificial muscle fiber may expand, and because of the twist in the structure of the artificial muscle fiber, the expansion transforms into torsional and/or tensile forces (i.e., actuation). An effective way of powering the artificial muscle fibers is by delivering thermal energy to the artificial muscle fiber through radiation or electrical conduction. However, the artificial muscle fiber may be powered with other methods such as photo absorption, chemical reactions, etc.

An artificial muscle fiber may be designed to create a desired actuation based on the specific application for which the artificial muscle fiber is designed. One way to control the actuation is by determining the twist in the artificial muscle fiber. The more twist in the artificial muscle fiber, the more actuation that may be generated upon actuation of the artificial muscle fiber.

An artificial muscle fiber may be comprised of, but not limited to, a polymer based fiber. For example, Nylon-6, Nylon-6,6, polyethylene, polyvinylidene fluoride, Nylon-6,10, Nylon-6,12, liquid crystalline polymers such as polyarylate, and combinations thereof may be included in the artificial muscle actuators. The artificial muscle fibers may also include carbon nanotube (CNT) based materials.

The specific characteristics of an artificial muscle fiber, such as width, material, twist, actuation, etc., may be established based on the specific application for which the artificial muscle fiber is designed.

In one or more embodiments of the invention, the artificial muscle fibers may be similar to artificial muscles (actuators) disclosed in U.S. patent application Ser. No. 14/610,905 filed Jan. 30, 2015, entitled “COILED AND NON-COILED TWISTED NANOFIBER YARN TORSIONAL AND TENSILE ACTUATORS.” The contents of this application are hereby incorporated by referenced in its entirety.

One or more embodiments of the invention provide methods for easy shaping artificial muscle fibers, and installing the artificial muscle fibers in actuator devices. Easy shaping and installing the artificial muscle fibers may provide less expensive manufacturing of the actuator devices.

Generally, designing and installing an electric motor or a part of an electric motor in an electromechanical system may be difficult because the electric motor must be tuned up for a desired actuation in the electromechanical system. For example, size, power, actuation, electrical and mechanical connections, installation, etc. (i.e., characteristics) of the electric motor must be tuned to comply with the electromechanical system. However, electric motors have specific characteristics determined by manufacturers, and it is difficult to modify these characteristics for a desired application.

Embodiments disclosed herein may facilitate producing an actuator device to desired properties. For example, it may be desired to modify an electromechanical system for a specific application. Rather than finding and purchasing an electric motor that complies with that application, embodiments disclose herein allow for developing the electric motor by shaping an artificial muscle fiber to a desired shape, and installing the artificial muscle fiber in the electromechanical system to create a desired actuation. In other words, known quantities of stock artificial muscle fibers may be combined with the customer's desired parameters to produce a specific desired actuator device in accordance with one or more embodiments disclosed herein.

In one or more embodiments of the invention, the artificial muscle fiber is shaped to the specific shapes (e.g., loops, hooks, etc.) and annealed to retain the specific shapes. The artificial muscle fiber may be annealed to a temperature at least above the glass transition temperature and below the melting temperature of the material of the artificial muscle fiber. The specific annealing temperature depends on the specific characteristics of the artificial muscle fiber, such as size dimensions of the artificial muscle fiber, materials used in the artificial muscle fiber, etc.

The specific shapes disclosed herein are not limited to loops and hooks, but may be any suitable shape such as spirals, squares, or non-regular shapes.

In one or more embodiments, a fastener may fasten the artificial muscle fiber to a device or a substrate. The fastener may pass through a specific shape of the artificial muscle fiber to fasten the artificial muscle fiber to the device or the substrate. The specific shape of the artificial muscle fiber may provide a mechanical or an electrical connection between the artificial muscle fiber and the substrate in accordance with one or more embodiments of the invention. Screws, nails, bolts, rivets, axels, rods, or other fasteners may be used to secure the artificial muscle fiber through the specific shape. In some embodiments, threads, fibers, or even glue may be used to help secure artificial muscle fiber to the substrate.

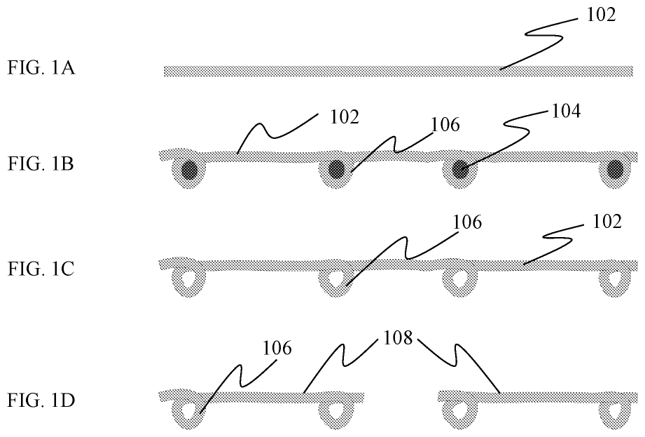

FIGS. 1A-1D are implementation examples in accordance with one or more embodiments of the invention that shows how an artificial muscle fiber may be formed into a desired shape. First, as shown in FIG. 1A, an artificial muscle fiber (102) is provided. Then, as shown in FIG. 1B, the artificial muscle fiber is tethered or wrapped around a plurality of shape-setting pieces (104) (e.g., rods) to form a plurality of loops (106) (an example of the specific shapes) along the length of the artificial muscle fiber (102). One of ordinary skill in the art will appreciate that the specific shapes are not limited to loops (106) and the shape-setting pieces (104) are not limited to rods, and different types of shape-setting pieces (104) may be used to form different forms of the specific shapes.

While the artificial muscle fiber (102) is tethered around the shape-setting pieces (104), the artificial muscle fiber (102) is annealed and then cooled down to retain the loops (106) without assistance of the shape-setting pieces (104). Then, as shown in FIG. 1C, the shape-setting pieces (104) may be removed from the artificial muscle fiber (102).

In one or more embodiments disclosed herein, the shape-setting pieces (104) may be used to anneal the artificial muscle fiber (102). For example, the shape-setting pieces (104) may be a type metal or another type of a heat conductor, and may be resistively heated and conduct the heat to the artificial muscle fiber (102). Annealing is not limited to the resistive heating of the shape-setting pieces, and other heating methods known in the art may be used to heat the shape-setting pieces (104) or the artificial muscle fiber (102). In one or more embodiments, a coating may be applied to the artificial muscle fiber (102) after the artificial muscle fiber (102) is annealed into a desired shape.

In FIG. 1D, the artificial muscle fiber (102) shown in FIG. 1C may be cut between the loops (106) to create artificial muscle segments (108) in accordance with one or more embodiments of the invention.

In one or more embodiments the artificial muscle segments may have at least two of the specific shapes for connections. The artificial muscle segments are not limited to including only two of the specific shapes and may include as many of the specific shapes as the application to which the artificial muscle segments are used in require.

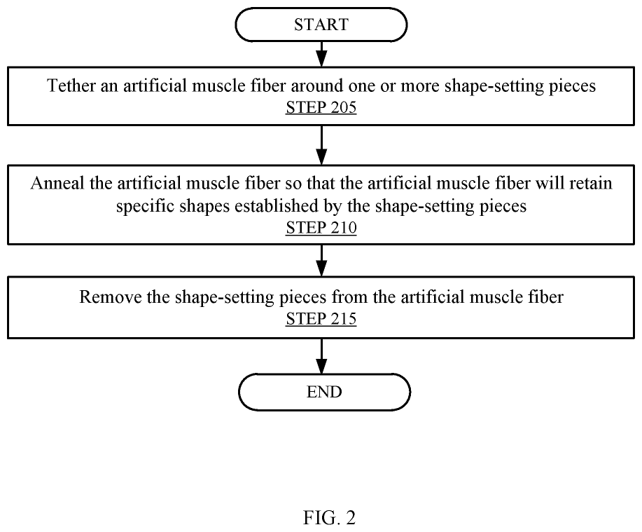

FIG. 2 is a flow chart in accordance with one or more embodiments of the invention. The flowchart depicts a method for manufacturing an artificial muscle fiber device (i.e., actuator device). In STEP 205, an artificial muscle fiber is tethered around one or more shape-setting pieces. For example, as shown in FIG. 1B, the artificial muscle fiber (102) is tethered around the shape-setting pieces (104) to form the loops (106) in the artificial muscle fiber (102).

The artificial muscle fiber may be tethered one or more rounds or less than one round (i.e., a fraction of a round) around any of the shape-setting pieces. It would have been apparent to one of ordinary skill in the art that the artificial muscle fiber may be disposed on the shape-setting pieces in other configurations.

In STEP 210, the artificial muscle fiber is annealed so that the artificial muscle fiber will retain specific shapes established by the shape-setting pieces. For example, the artificial muscle fiber (102) shown in FIG. 1B is annealed to retain the loops (106). In one or more embodiments of the invention, the artificial muscle fiber may be annealed to a temperature at least above the glass transition temperature and below the melting temperature of the material of the artificial muscle fiber. The specific annealing temperature depends on the specific characteristics of the artificial muscle fiber, such as size dimensions of the artificial muscle fiber, materials used in the artificial muscle fiber, etc.

In STEP 215, the shape-setting pieces are removed from the artificial muscle fiber. For example, as shown in FIG. 1C, the shape-setting pieces (104) shown in FIG. 1B are removed from the artificial muscle fiber (102). In one or more embodiments, the shape-setting pieces may be removed from the artificial muscle fiber after the artificial muscle fiber cools down to a temperature below the glass transition temperature of the material of the artificial muscle fiber. In these embodiments, when the artificial muscle fiber reaches a temperature below the glass transition temperature, the artificial muscle fiber becomes more rigid and retains the specific shapes. However, it would have been apparent to one of ordinary skill in the art that the shape-setting pieces may be removed from the artificial muscle fiber while the artificial muscle fiber has a temperature at or above the glass transition temperature of the material of the artificial muscle fiber.

In one or more embodiments of the invention, the artificial muscle fiber may be cut to form desired pieces of the artificial muscle fiber with desired sizes. In one or more embodiments of the invention, the artificial muscle fiber, may be cut before or after the artificial muscle fiber reaches a temperature below the glass transition temperature. The artificial muscle fiber may be cut from a location of the artificial muscle fiber that is between two adjacent specific shapes to create artificial muscle segments. For example, as shown in FIG. 1D, the artificial muscle fiber (102) shown in FIG. 1C may be cut to create two artificial muscle segments (108).

In one or more embodiments of the invention, any of the specific shapes may be cut to form a shape different from that specific shape. For example, the loop (106) shown in FIG. 1C may be cut to change the loop (106) into a hook.

In one or more embodiments of the invention, the artificial muscle fibers/segments that include the specific shapes may be developed into an artificial muscle fiber device by fastening the artificial muscle fibers/segments to a holder (i.e., substrate). In an example in accordance with one or more embodiments of the invention, the artificial muscle fibers/segments may be fastened to the holder by passing screws through the empty space of the loops and screwing the screws to the holder.

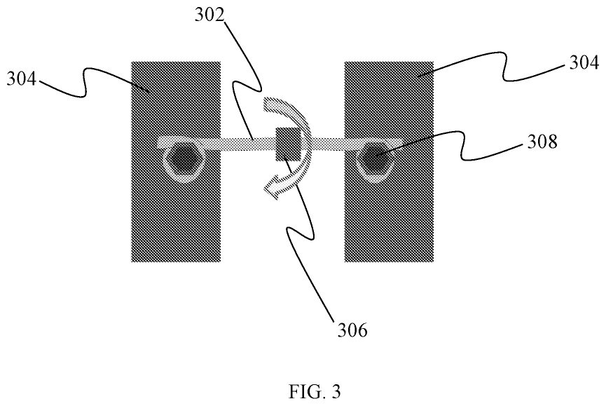

FIGS. 3-5 show implementation examples of artificial muscle fiber devices that include artificial muscle segments in accordance with one or more embodiments of the invention. It would have been apparent to one of ordinary skill in the art that these examples are for better understanding of the embodiments of the invention, and the invention is not limited to these examples.

FIG. 3 shows an artificial muscle fiber device in accordance with one or more embodiments of the invention that has an artificial muscle segment (302) fastened to holders (304) using fasteners (308). The end portions of the artificial muscle segment (302) have a specific shape of a loop, and the fasteners (308) connect the artificial muscle segment (302) to the holders (304) through the loops.

In one or more embodiments of the invention, upon powering the artificial muscle segment (302), the artificial muscle segment (302) creates an actuation (e.g., torsional or tensional forces). For example, the artificial muscle segment (302) may create torsional forces and rotate as shown by the arrow in FIG. 3. The torsional forces may also rotate the holders (304) with respect to each other. In another example, the artificial muscle segment (302) may create tensional forces that may move the holders (304) along the length of the artificial muscle segment (302).

In accordance with one or more embodiments of the invention as shown in FIG. 3, a secondary device (306) may be disposed on the artificial muscle segment (302) and between the loops. Upon actuation of the artificial muscle segment (302), the secondary device (306) may be moved in any direction (e.g., may rotate) that the artificial muscle segment (302) is designed to create the actuation in that direction. In one or more embodiments of the invention, the secondary device may be a camera, a light source, etc.

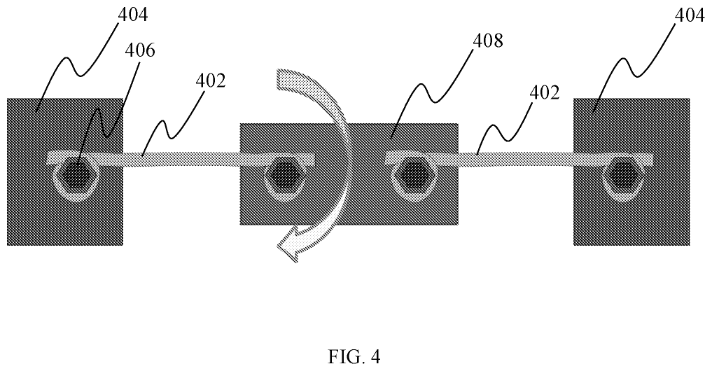

FIG. 4 shows another implementation example in accordance with one or more embodiments of the invention. As shown in FIG. 4, two artificial muscle segments (402) are fastened to two holders (404) and a substrate (408) using fasteners (406). The artificial muscle segments (402) are fastened to hold the substrate (408) between the holders (404). Upon actuation of the artificial muscle segments (402), the artificial muscle segments (402) create an actuation that may move the substrate (408). For example, the artificial muscle segments (402) may have been designed to create torsional forces that rotate the substrate (408) as shown by the arrow in FIG. 4.

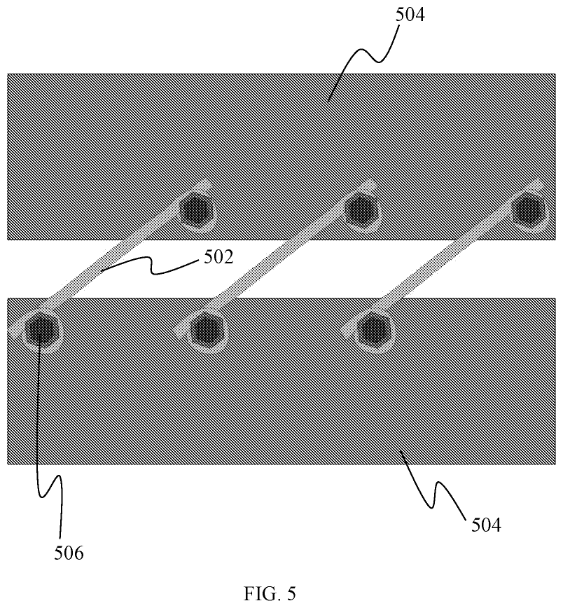

FIG. 5 shows another implementation example in accordance with one or more embodiments of the invention. FIG. 5 shows a hinge-type artificial muscle fiber device that has three of artificial muscle segments (502) attached to two paddles (504) using a plurality of fasteners (506). Actuation of the artificial muscle segments (502) may result in the paddles (504) to open or close relative to each other. It would have been apparent to one of ordinary skill in the art that the number of the artificial muscle segments (502) is not limited to three and may be any other number.

In one or more embodiments of the invention, the artificial muscle segments (502) may be disposed in parallel with each other. The artificial muscle segments (502) may be disposed with an offset with respect to each other that may be in a direction perpendicular to a gap between the paddles (504). It would have been apparent to one of ordinary skill in the art that the artificial muscle segments (502) may be disposed in other configurations relative to each other.

It would have been apparent to one of ordinary skill in the art that the artificial muscle segments disclosed herein may be fastened to a substrate/holder or a device in a way based on a specific application. For example, in one or more embodiments, the artificial muscle segments may be loosely fastened to a holder so that the specific shape of the artificial muscle segment can rotate around the fastener. In a more specific example of one or more embodiments of the invention, an artificial muscle segment may have a loop-shape end (i.e. specific shape), and a screw (i.e., fastener) may fasten the end of the artificial muscle segment to a holder. In this example, the screw may pass through the loop-shape end and may fasten the loop-shape end to the holder so that the loop-shape end can freely rotate around the screw.

Alternatively, the artificial muscle segment may be tightly fastened so that the specific shape of the artificial muscle segment is fixed from any movement around the fastener. For example, the screw may tightly fasten the loop-shape end to the holder to prevent rotation of the loop-shape end around the screw.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

1. A method of manufacturing an artificial muscle fiber device, the method comprising:
tethering an artificial muscle fiber around one or more shape-setting pieces;
annealing the artificial muscle fiber so that the artificial muscle fiber will retain specific shapes established by the shape-setting pieces; and
removing the shape-setting pieces from the artificial muscle fiber.

2. The method of claim 1, further comprising: heating the artificial muscle fiber through the shape-setting pieces during annealing.

3. The method according to claim 1, wherein the shape-setting pieces are in an even number that is not less than four, and the method further comprises:
after annealing, cutting the artificial muscle fiber to create two artificial muscle segments,
wherein each of the segments has two or more of the specific shapes.

4. The method according to claim 1, further comprising:
fastening the artificial muscle fiber to one or more holders through the specific shapes using fasteners.

5. The method according to claim 1, wherein the artificial muscle fiber is annealed at a temperature greater than glass transition temperature of the artificial muscle fiber and less than melting temperature of the artificial muscle fiber.

6. The method according to claim 1, wherein the artificial muscle fiber comprises a polymer fiber selected from a group consisting of Nylon 6, Nylon 6,6, polyethylene, polyvinylidene fluoride, Nylon 6,10, Nylon 6,12, liquid crystalline polymers, polyarylate, and combinations thereof.

7. The method according to claim 1, wherein the artificial muscle fiber comprises carbon nanotubes.

8. The method according to claim 1, wherein the shape-setting pieces are rods, and the specific shapes are loops or hooks.

9. An artificial muscle actuator device comprising:
a first artificial muscle fiber segment comprising:
a first specific shape,
wherein the first specific shape is established by first segment shape-setting pieces,
wherein the first specific shape is established by annealing the first specific shape,
a first fastener; and
a first holder,
wherein the first fastener fastens the first specific shape to the first holder.

10. The device of claim 9, further comprising:
a second holder; and
a second fastener,
wherein the first artificial muscle fiber segment comprises:
a second specific shape,
wherein the second specific shape is established by the first segment shape-setting pieces,
wherein the second specific shape is established by annealing the second specific shape, and
wherein the second fastener fastens the second specific shape to the second holder.

11. The device of claim 10, further comprising:
a second artificial muscle fiber segment comprising:
a third specific shape and a fourth specific shape,
wherein the third and fourth specific shapes are established by second segment shape-setting pieces,
wherein the third and fourth specific shapes are established by annealing the third and fourth specific shapes, respectively,
a third holder;
a third fastener that fastens the third specific shape to the second holder; and
a fourth fastener that fastens the fourth specific shape to the third holder.

12. The device of claim 11, wherein actuations of the first and second artificial muscle fiber segments rotate the second holder.

13. The device of claim 10, further comprising:
a second artificial muscle fiber segment comprising:
a third specific shape and a fourth specific shape,
wherein the third and fourth specific shapes are established by second segment shape-setting pieces,
wherein the third and fourth specific shapes are established by annealing the third and fourth specific shapes,
a third fastener that fastens the third specific shape to the first holder; and
a fourth fastener that fastens the fourth specific shape to the second holder,
wherein the first and second artificial muscle fiber segments are parallel, and
wherein the second artificial muscle fiber segment is shifted from the first artificial muscle fiber segment.

14. The device of claim 13, wherein the second artificial muscle fiber segment is shifted from the first artificial muscle fiber segment in a direction perpendicular to a gap between the first and second holders.

15. The device according to claim 9,
wherein the first and second segment shape-setting pieces are similar,
wherein the first, second, third, and fourth specific shapes are similar, and
wherein the first, second, third, and fourth fasteners pass through the first, second, third, and fourth specific shapes, respectively.

16. The device according to claim 9,
wherein the first and second segment shape-setting pieces are rods,
wherein the first, second, third, and fourth specific shapes are loops, and
wherein the first, second, third, and fourth fasteners pass through the first, second, third, and fourth specific shapes, respectively.

17. The device according to claim 10, further comprising:
a secondary device fixed on the first artificial muscle fiber segment between the first and second specific shapes,
wherein actuation of the first artificial muscle fiber segment rotates the secondary device.

18. The device according to claim 9, wherein the first, second, third, and fourth specific shapes are hooks.

19. The device according to claim 9, wherein at least one of the artificial muscle fiber segments comprise carbon nanotubes.

20. The device according to claim 9, wherein at least one of the artificial muscle fiber segments comprise a polymer fiber selected from a group consisting of Nylon 6, Nylon 6,6, polyethylene, polyvinylidene fluoride, Nylon 6,10, Nylon 6,12, liquid crystalline polymers, polyarylate, and combinations thereof.
